(19) United States
(12) Patent Application Publication
(10) Pub. No.: US 2007/0231881 A1
(43) Pub. Date: Oct. 4, 2007

(54) Biomolecular interaction analyzer

(76) Inventors: Tetsuro Miyamoto, Kasumigaura (JP); Mami Hakari, Mito (JP); Shigenori Togashi, Abiko (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/528,518

(22) Filed: Sep. 28, 2006

(30) Foreign Application Priority Data
Mar. 30, 2006 (JP) 2006-092749

(51) Int. Cl. C12M 1/34; G06T 7/40

(52) U.S. Cl. 435/287.2; 356/445; 382/120

(57) ABSTRACT

Obtained is a biomolecular interaction analyzer which is capable of simultaneously measuring a large number of many kinds of samples. The biomolecular interaction analyzer is an analyzer for measuring biomolecular interactions, which includes a measurement chip and a color CCD array. The measurement chip includes a plurality of measurement areas in each of which a fine particle sensor coated with a noble metal is formed. In the color CCD array, two-dimensionally arrayed light receiving elements respectively for measuring optical properties of the measurement areas are utilized.

### CLAIM OF PRIORITY

The present application claims priority from Japanese application JP 2006-92749 filed on Mar. 30, 2006, the content of which is hereby incorporated by reference into this application.

### BACKGROUND OF THE INVENTION

1. Field of the Invention

The present invention relates to an analyzer for analyzing molecular interactions, which adopts a biosensor, the analyzer being used for biochemical study, pharmaceutical development, medical diagnosis, and food inspection.

2. Description of the Prior Art

Conventional sensors of this type include a sensor in which Surface Plasmon Resonance (SPR) is utilized. The surface plasmon is a wave of condensation and rarefaction of free electrons which travel on an interface between metallic thin film and a dielectric. Since the surface plasmon is largely influenced by a dielectric constant at the interface, it is used as a detection principle for an immunosensor, a gas sensor and the like.

FIG. 7 shows a specific example of a structure of an analyzer which is applied to the above sensor.

A noble metal film 72, which is made of such as gold or silver, is formed on a surface of a transparent support 71 having high refractive index, such as a prism. A molecular recognition layer 73 is formed on the thin film 72. A parallel light 74 is irradiated on the film 72 from a prism side by using a light source 75 so as to excite a surface plasmon of the film 72. Under condition of total reflection, a regular reflection light 77 is detected by a detector 78 while varying an incidence angle 76. By this way, an excitation of the surface plasmon can be recognized. That is, at a resonant incident angle 79, since energy of an incident light is consumed for the excitation of the surface plasmon, the intensity of a reflected light is reduced to an extreme extent. When target biomolecules are captured on the molecular recognition layer 73, the intensity of the reflected light is reduced to an extreme extent at a resonant incident angle 80. A dielectric constant of the molecules existing on the metal surface formed on the molecular recognition layer 73 can be specified by knowing a resonance angle because the resonance angle is sensitively dependent on the dielectric constant in an area several hundreds nm or less away from the interface. For this way, the Surface Plasmon Resonance can be utilized as a sensor.

For example, a structure, which recognizes particular molecules and causes molecular bonds thereto, is previously formed on the surface of the film 72. When the bonds with the particular molecules occur to the structure, the dielectric constant is varied. It is therefore possible to immediately know that the particular molecules are captured on the molecular recognition layer 73 by monitoring the reflected light at a reflection angle corresponding to the molecules.

In comparison with the Surface Plasmon Resonance sensor, as a sensor which is capable of performing measurement using a more simple optical system, Japanese Unexamined Patent Application Publication No. 2000-55920 describes a use of a noble metal fine particle sensor.

FIG. 8 shows a structure of the noble metal fine particle sensor. A layer of fine particles 83 of polymer, SiO₂, TiO₂, or the like, is formed on a noble metal film 82 on a substrate 81, and then, by performing evaporation or sputter deposition of a noble metal such as gold, silver, copper, and platinum, cap-shaped fine particles 84 of gold, silver, copper, and platinum is formed on the fine particle 83 (Japanese Unexamined Patent Application Publication No. Hei 11-1703).

A formation of the noble metal fine particles causes the substrate to be outstandingly colored (Japanese Unexamined Patent Application Publication No. Hei 10-339808). A color phenomenon is caused by the fact that light having a particular range of wavelength is absorbed when white light is reflected. The noble metal fine particles can be utilized as a principle for detecting reactions, in which a refraction index of a surface is varied, because an absorption peak wavelength of the noble metal fine particle depends on the refraction index of the surface (Japanese Unexamined Patent Application Publication No. Hei 11-326193). Moreover, the noble metal fine particle can also be utilized as a biosensor by modifying its surface with a biomolecule having unique absorption ability, such as an antibody and DNA (Japanese Unexamined Patent Application Publications No. 2000-55920 and No. 2002-365210).

### SUMMARY OF THE INVENTION

In order to measure a resonant incident angle in the analyzer of the prior art, in which the surface plasmon sensor is utilized, it is necessary to maintain a positional relationship among a light source of an irradiation light, a metal thin film, and a light detector with high accuracy, and to drive them. Additionally, it is necessary to control or correct temperatures of a sample to be measured and of the analyzer as a whole because the surface plasmon resonance method is sensitive to temperature.

In spite of an increasing need for such a measurement of the interaction between biomolecules in recent years, the down-sizing of the analyzer and the parallelization thereof for the purpose of detecting a plurality of samples have been difficult to achieve.

It is an object of the present invention to provide a biomolecular interaction analyzer which makes it possible to simultaneously perform measurement on a large number of different kinds of objects to be measured.

In order to solve the above problems, the present invention provides an analyzer for measuring biomolecular interactions and includes a measurement chip having a plurality of measurement areas in which fine particle sensors coated with a noble metal are formed, and a color CCD array in which two-dimensionally arrayed light receiving elements for measuring optical properties of the measurement area are utilized.

The present invention makes it possible to simultaneously measure interactions among a plurality of samples to be measured in a plurality of sensor areas by means of an optical device such as a CCD array sensor. The present invention also makes it possible to prevent a sample from being contaminated and to facilitate a preparation by separating the sensor and the samples to be measured depending on each measurement condition and by achieving a simple change thereof, thus shortening time taken for the measurement of a large number of samples under many kinds of conditions.

### BRIEF DESCRIPTION OF THE DRAWINGS

FIG. 1 is a plan view of a measurement chip of one embodiment according to the present invention.

FIG. 2 is an enlarged view of FIG. 1.

FIG. 3 is a block diagram of an analyzer of a first embodiment according to the present invention.

FIG. 4 is a graph showing an analysis example according to the first embodiment.

FIG. 5 is a plan view of a measurement chip according to a second embodiment.

FIG. 6 is a block diagram of the analyzer according to the second embodiment.

FIG. 7 is a schematic view showing an analyzer to which a sensor according to the prior art is applied.

FIG. 8 is a side view showing a structure of a noble metal fine particle sensor according to the prior art.

### DETAILED DESCRIPTION OF THE PREFERRED EMBODIMENTS

The descriptions are made below of a structure of a chip which includes a plurality of sensor areas each having a fine particle of a noble metal, and of a structure of an analyzer in which two-dimensionally arrayed light receiving elements are utilized.

### Example 1

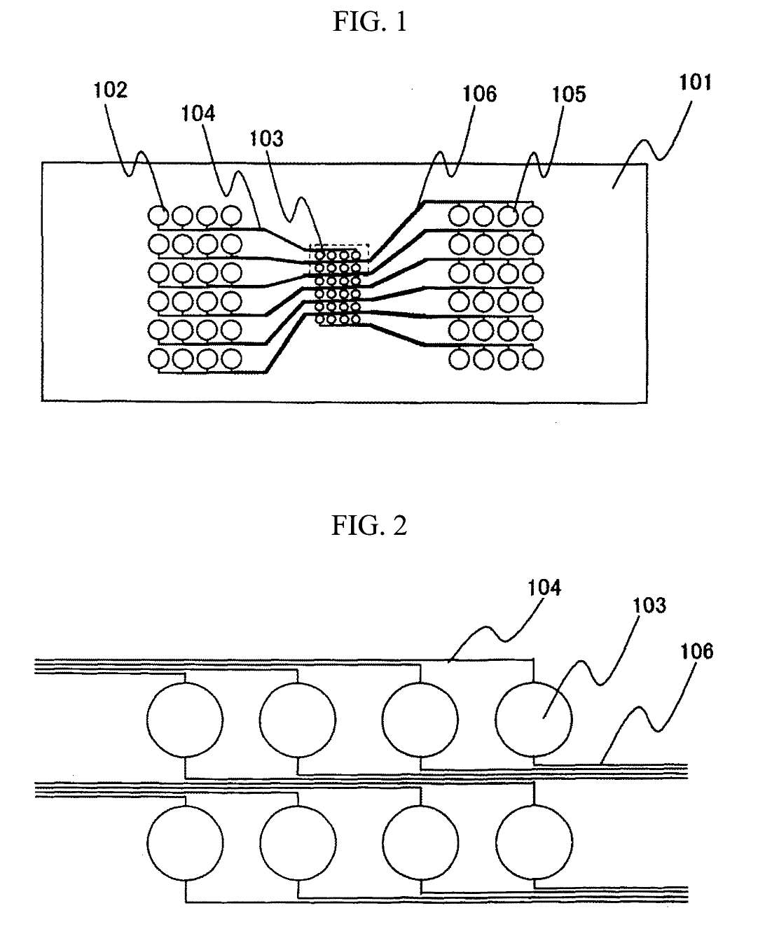

A first example according to the present invention is described with reference to FIGS. 1 to 4.

FIG. 1 shows a schematic structure of a measurement chip according to the present invention. The measurement chip 101 includes a plurality of sample solution holding areas 102, a plurality of measurement areas 103 and a plurality of measured sample solution holding areas 105. The sample solution holding areas 102, which are disposed in a two-dimensional array, temporarily hold sample solutions to be measured. The measurement areas 103, which are disposed in a two-dimensional array, have noble metal fine particle sensors formed therein, and the measured sample solution holding areas 105, which are disposed in a two-dimensional array, hold measured sample solutions after measurement. The sample solution holding areas 102 are respectively connected to the measurement areas 103 via corresponding injection flow paths 104, and the measurement areas 103 are respectively connected to the measured sample holding areas 105 via corresponding discharge flow paths 106. FIG. 2 shows an enlarged view of a part enclosed by a dashed line in FIG. 1.

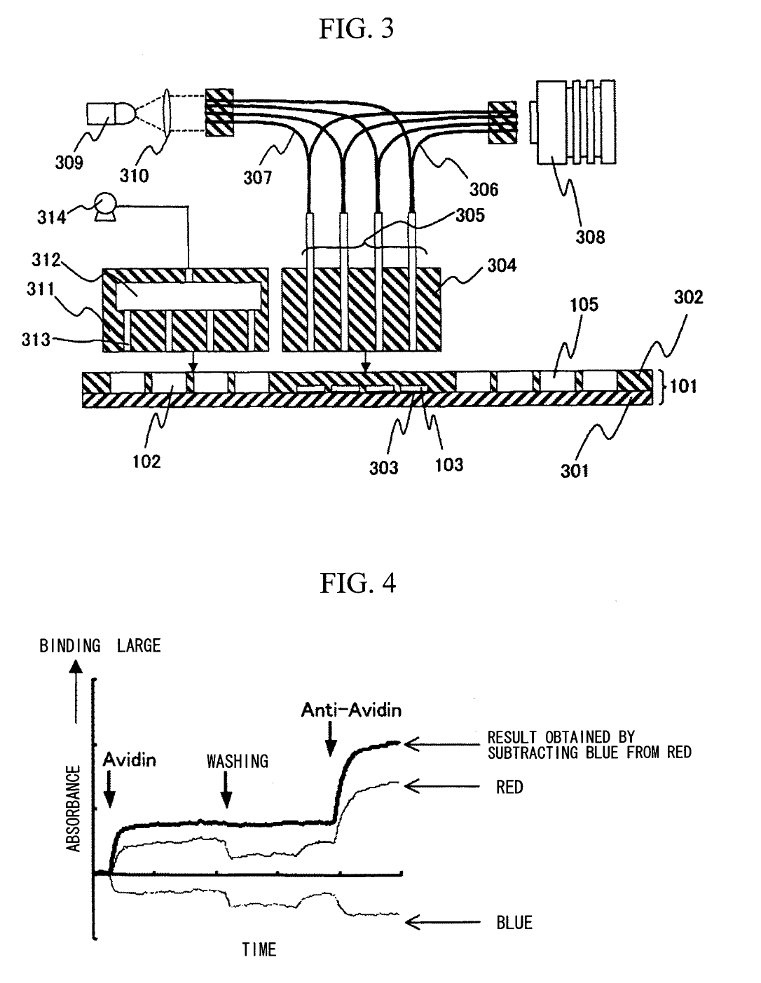

FIG. 3 shows a cross-section of the measurement chip 101 and a schematic structure of the analyzer. The measurement chip 101 includes a measurement chip substrate 301 and a top board 302 of a measurement chip. On the measurement chip substrate 301, sensors 303 having noble metal fine particles of a particle size of about 100 nm, are formed in positions corresponding to the measurement areas 103. In the top board 302 of the measurement chip, the measurement sample holding areas 102, the measurement areas 103, the measured sample holding areas 105, the injection flow paths 104 (not shown in the figure), and the discharge flow paths 106 (not shown in the figure) are formed. The areas 102 for holding the samples to be measured and measured sample holding areas 105 are open on a side of the top board 302 so as to enable the injection and discharge of the sample solutions. The top board 302 of the measurement chip preferably is made of a material having a high optical transparency, such as quartz glass, polystyrene resin, and silicon resin so as to avoid the interference in an optical measurement described below.

A measurement probe 304 is disposed on the side of the top board 302 of the measurement chip 101. The measurement probe 304 includes optical fiber arrays 305 for measurement which are two-dimensionally disposed thereto in a manner where the optical fiber arrays 305 respectively correspond to the measurement areas 103. One side of each of the optical fiber arrays 305 are disposed facing the measurement areas 103, and the other side thereof is branched into optical fibers 306 for measurement and optical fibers 307 for a light source. An end of each of the optical fibers 306 for measurement is disposed in an array pattern in a manner that the end faces a light receiving surface of a color CCD array 308. Parallel light, which has traveled from a white light source 309 through a lens 310, enters an end of the optical fibers 307 for the light source. The parallel light, which has entered the fibers 307, enters the measurement areas 103. A part of reflected light travels through the optical fibers 306 for measurement, and then enters the color CCD array 308 in an array pattern, and is converted to R, G, and B light intensity signals. These signals are recorded in a data processing device (not shown in a figure). It is conceivable that the color CCD array 308 is capable of performing measurement in at least two or more different ranges of wavelength.

Moreover, a pressure probe 311 is disposed in a position corresponding to the measurement sample holding areas 102 on the side of the top board 302 of the measurement chip 101. The pressure probe 311 includes a pressure chamber 312, and pressure flow paths 313 which respectively correspond to the measurement sample holding areas 102. The pressure chamber 312 is connected with a pressure pump 314, so as to provide the pressure chamber 312 with compressed air.

Focusing on a case where bindings between a plurality of antigen samples and a plurality of antibody samples are measured, measurement of interactions will be described.

Samples of antibodies, which are to be measured for binding, are respectively fixed to the noble metal fine particle sensors 303 in the corresponding measurement areas 103. A method of the fixing may preferably be a method in which antibodies are respectively added dropwise to the noble metal fine particle sensors 303 before unifying the measurement chip top board 302 and the measurement chip substrate 301, or may be a method in which antibody solutions are injected from the measurement sample holding areas 102 on the measurement chip 101 respectively to the noble metal fine particle sensors 303 through the corresponding injection flow paths 104, respectively. Then, a buffer solution is filled in the measurement areas 103 and in the injection flow paths 104.

Subsequently, antigen solutions, which are also to be measured, are injected respectively into the measurement sample holding areas 102 on the measurement chip 101.

Next, as shown in FIG. 3, the measurement chip 101 is set to the analyzer to start an optical measurement. The pressure probe is contacted with the measurement chip 101 and air pressure is applied to each of the measurement sample holding areas 102 by means of the pressure pump 314. Thereby, the antigen solutions previously injected as described above are injected respectively from the measurement sample holding areas 102 to the measurement areas 103 through the corresponding injection flow paths 104. In a case where the antibodies, which have previously been fixed to the noble metal fine particle sensors 303, are respectively bound with the antigens injected thereon, optical properties of each of the sensors are changed. Moreover, this change in each of the sensors causes a change in wavelength obtained by passing reflected light through a spectrometer, and also causes a change in the shape of wavelength spectrum which indicates the intensity of light of the wavelength. When the particle size of each of the noble metal fine particle sensors 303 is about 100 nm, the absorption peak of the reflected light is about 550 nm. In a case where a binding is occurred, it is known that the peak wavelength is shifted to the long wavelength side. At this time, increase and decrease in the intensity of the light are occurred in the reverse order between the areas of blue (B) light and red (R) light, the blue (B) light being on the shorter wavelength side (about no more than 400 nm) than green (G) light which is in the peak wavelength range, and the red (R) light being on the longer wavelength side (about no less than 600 nm) than green (G) light. Since the variations in the light intensity are correlated with the amount of binding, the difference is obtained between blue and red to improve the S/N ratio of each signals, and then the improved signals is recorded respectively as binding signals.

FIG. 4 shows the binding signals between Avidin (antigen) and Anti-Avidin (antibody). By obtaining the difference in the signal intensity between red and blue, it can be seen that a binding is occurred and causes the increase in the signal intensity when adding the Anti-Avidin (antibody).

As described above, the interactions of the different combinations of antigen-antibody from each other can be simultaneously and easily measured. Note that, the number of samples which can be measured depends on the number of the areas formed on the chip and the number of pixels of the light receiving element. Accordingly, a very large number of samples can be measured.

### Example 2

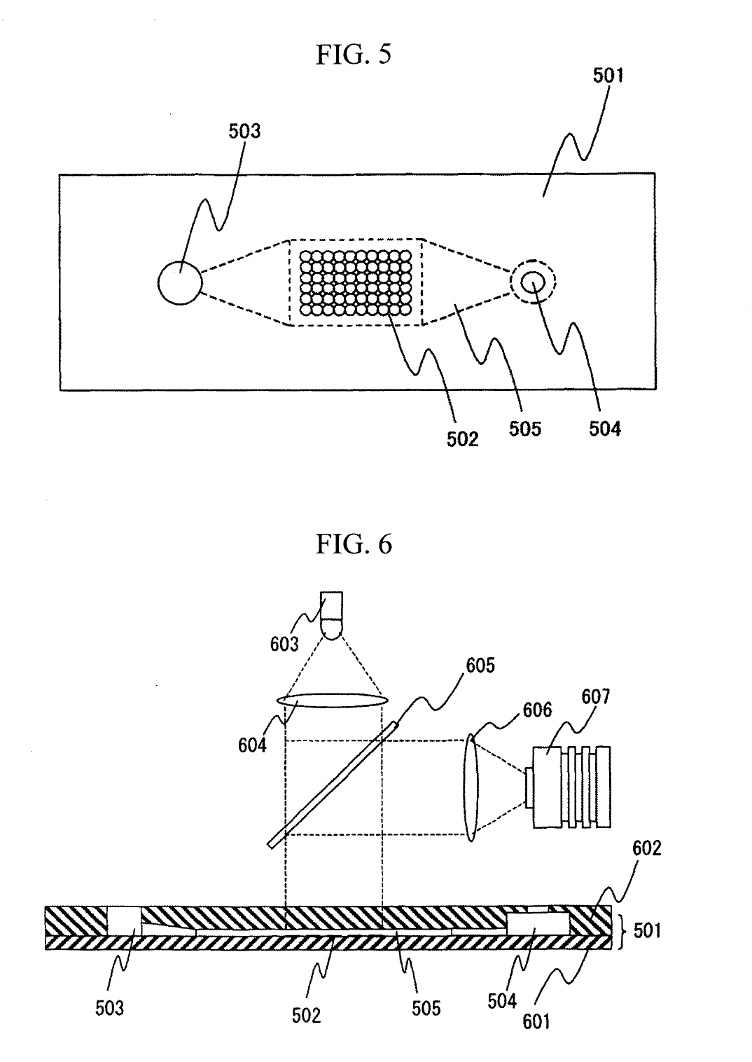

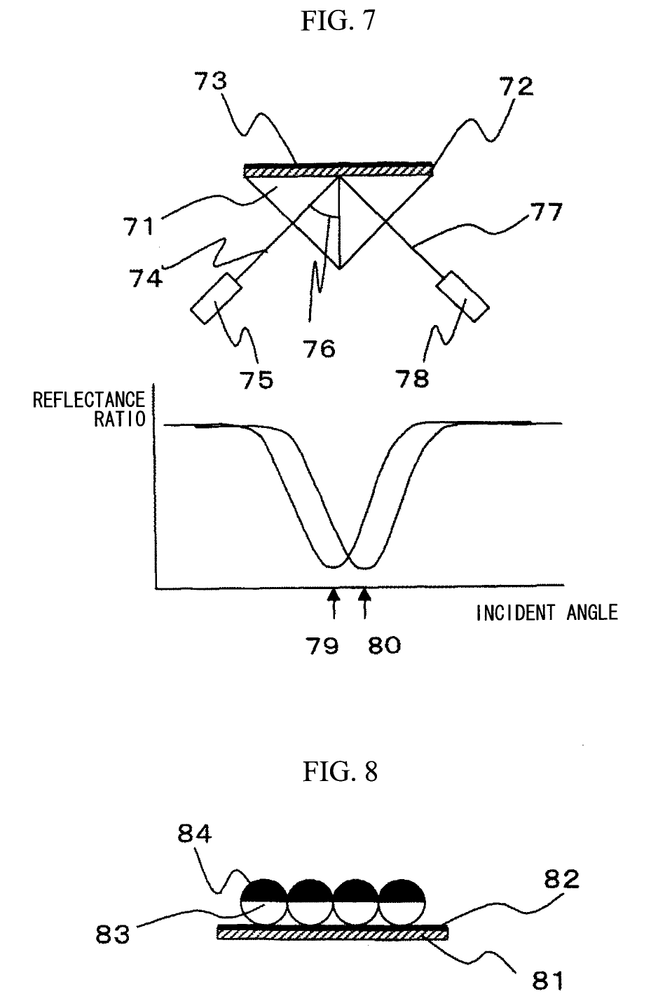

A second example will be described with reference to FIGS. 5 and 6.

FIG. 5 shows a schematic structure of a measurement chip. A measurement chip 501 includes an area 503 for temporarily holding measurement sample solution, a flow path 505 in which a plurality of noble metal fine particle sensor areas 502 are formed in an array pattern, and an area 504 for holding the measured sample solution after measurement. The sample solution holding area 503 and the measured sample solution holding area 504 are connected with each other via the flow path 505.

FIG. 6 shows a schematic construction of an interaction analyzer. As in Example 1, the measurement chip 501 includes a measurement chip substrate 601 and a measurement chip top board 602. On the measurement chip substrate 601, the noble metal fine particle sensor areas 502 are formed, and on the measurement chip top board 602, the sample solution holding area 503, the measured sample solution holding area 504, and the flow path 505 are formed. Moreover, as in Example 1, the measurement chip top board 602 preferably is made of a material with high optical transparency.

A plurality of different kinds of the biomolecules such as antigen are previously fixed respectively to the noble metal fine particle sensor areas 502 formed in an array pattern. A measurement sample solution is added dropwise to the sample solution holding area 503. Subsequently, as in Example 1, when air pressure is applied to the area 503 by means of the pressure probe (not shown in the figure), then the measurement sample solution is transferred through the flow path 505 and then contacts with each of the noble metal fine particle sensor areas 502, and the measurement sample solution then reacts with the fixed biomolecules. At this time, reflected light of light, which has entered the noble metal fine particle sensor areas 502 from a white light source 603 through a lens 604, is transmitted through a beam splitter 605 and the lens 604. Then the reflected light enters a color CCD array. Measurement of the interaction, such as an antigen-antibody reaction, can thus be performed as in Example 1.

This embodiment does not allow simultaneous measurement, such as that between many kinds of antigens and many kinds of antibodies, as in Example 1. However, for example, as in a case of a blood test related to allergies, when it is required to know reactions of many kinds of antibodies with one kind of a sample solution to be tested, this embodiment is capable of simultaneously measuring many kinds of antibodies with an analyzer having a relatively simple structure.

What is claimed is:

1. A biomolecular interaction analyzer comprising:
a measurement chip having a plurality of measurement areas in each of which a fine particle sensor coated with a noble metal is formed; and
a color CCD array in which two-dimensionally arrayed light receiving elements respectively for measuring optical properties of the measurement areas are utilized.

2. The biomolecular interaction analyzer as set forth in claim 1, wherein
the color CCD array is capable of measuring at least two or more different ranges of wavelength.

3. The biomolecular interaction analyzer as set forth in claim 1, wherein
the measurement chip includes a measurement sample holding area for temporarily holding sample solution to be measured, a measured sample holding area for holding a measured sample solution, injection flow paths connecting the measurement sample holding area with the corresponding measurement areas, and discharge flow paths connecting the corresponding measurement areas with the measured sample holding area.

4. The biomolecular interaction analyzer as set forth in claim 1, wherein
the measurement chip includes, a plurality of measurement sample holding areas for temporarily holding sample solutions to be measured, a plurality of measured sample holding areas for holding measured sample solutions, injection flow paths respectively connecting the measurement sample holding areas with the corresponding measurement areas, and discharge flow paths respectively connecting the measurement areas with the corresponding measured sample holding areas; and
the measurement sample holding areas and the measured sample holding areas are connected one-to-one with each other.

5. The biomolecular interaction analyzer as set forth in claim 1, wherein
the optical properties of the measurement area are measured via optical fibers for measurement by means of the color CCD array.
